(19) United States
(12) Patent Application Publication
(10) Pub. No.: US 2023/0233572 A1
(43) Pub. Date: Jul. 27, 2023

(54) OPHTHALMIC COMPOSITIONS COMPRISING A COMBINATION OF FLUOROQUINOLONE ANTIBACTERIAL AGENT AND AN ANTI-INFLAMMATORY AGENT

(71) Applicant: SENTISS PHARMA PRIVATE LIMITED, NEW DELHI (IN)

(72) Inventors: KOSHY PRAVEEN VARGHESE, GURUGRAM, HARYANA (IN); SUDHEER DASARIPALLI, GURUGRAM, HARYANA (IN); GUNEET SINGH RANDHAWA, GURUGRAM, HARYANA (IN); TARUN KUMAR MANDAL, GURUGRAM, HARYANA (IN); MALLINATH HARWALKAR, GURUGRAM, HARYANA (IN); KISHOR DEO, GURUGRAM, HARYANA (IN); DEEPAK BAHRI, GURUGRAM, HARYANA (IN)

(21) Appl. No.: 18/009,347

(22) PCT Filed: May 14, 2021
(86) PCT No.: PCT/IB2021/054152

§ 371 Date: Dec. 9, 2022

(30) Foreign Application Priority Data
Jun. 11, 2020 (IN) 202011024504

(51) Int. Cl. A61K 31/5383; A61K 31/407; A61K 9/00; A61P 31/04; A61P 27/02; A61K 47/18; A61K 47/32; A61K 47/40

(52) U.S. Cl. CPC A61K 31/5383; A61K 31/407; A61K 9/0048; A61P 31/04; A61P 27/02; A61K 47/186; A61K 47/183; A61K 47/32; A61K 47/40

(57) ABSTRACT

The present invention relates to an ophthalmic pharmaceutical composition comprising a combination of fluoroquinolone antibacterial agent and an anti-inflammatory agent along with a complexing agent and water. The composition is having a pH between 4 and 8. The present invention also relates to the methods of making an ophthalmic pharmaceutical composition and there uses thereof. The present invention also provides a method of treating and/or preventing an ophthalmic condition in a patient.

### FIELD OF THE INVENTION

The present invention provides an ophthalmic pharmaceutical composition comprising a combination of fluoroquinolone antibacterial agent and an anti-inflammatory agent along with a complexing agent and water, wherein the composition preferably having a pH between 4 and 8. Further, the present invention also relates to the methods of making an ophthalmic pharmaceutical composition as described herein and using the same.

The present invention also provides a method of treating and/or preventing an ophthalmic condition in a patient.

### BACKGROUND OF THE INVENTION

Inflammatory reactions can cause tissue damage, promoting scar formation in the eyes and its appendages, this condition can lead to undesired functional impairments. For these reasons, post cataract and other eye surgeries, medications such as anti-inflammatory drugs are often administered to aid in the healing process. To prevent this, the anti-inflammatory drugs are typically delivered to the ocular surface and the front of the eye in the form of eye drops.

In surgical treatment of the anterior segment of the eye, for example, in cataract surgery, post-operative inflammation can be reduced by the pre-operative and post-operative use of NSAIDs administered to the eye. There is a correlation between the blepharoconjunctival microbial flora present in the pre-operative stage of cataract Surgery and the infectious agents isolated from post-operative endophthalmitis. For this reason, it is common practice to sterilize or disinfect the external eye structure, both prior to surgery and post-operatively, until there is no longer a possibility of infection in the surgical wound, by treatment with an antibiotic. Therefore, a combination of suitable antibiotics either a steroid or an anti-inflammatory drug such as NSAIDs are useful for the treatment of eye and/or ear infections and ear injuries.

The results of cataract surgery depend on the surgeon's technique as well as the patient's healing in the postoperative period. By using antibiotics to stave off infection, corticosteroids to modulate healing and NSAIDs to quell inflammation, we can ensure optimal visual outcomes for our cataract surgery patients. The three primary classes of medications used around the time of cataract surgery are fluoroquinolone antibacterial agent, corticosteroids and nonsteroidal anti-inflammatory drugs (NSAIDs).

Levofloxacin is one of new fluoroquinolone antibacterial agent that develop the antibacterial activity by inhibition of DNA gyrase and topoisomerase IV and has been widely used against a broad spectrum of Gram-positive and Gram-negative ocular pathogens. In United States, because of a broad antibacterial spectrum of levofloxacin and its excellent antibacterial activity, a 0.5% (w/v) levofloxacin ophthalmic solution (Quixin 0.5%) has been widely used as an antibacterial ophthalmic solution comprising levofloxacin as an active ingredient. The composition-of-matter patent for levofloxacin (U.S. Pat. No. 5,053,407) expired on 20 Dec. 2010.

Ketorolac tromethamine is a nonsteroidal anti-inflammatory drug which, when administered systemically, has demonstrated analgesic, anti-inflammatory, and anti-pyretic activity. The mechanism of its action is thought to be due to its ability to inhibit prostaglandin biosynthesis.

ACULAR® ophthalmic solution (Ketorolac 0.5%) is indicated for the temporary relief of ocular itching due to seasonal allergic conjunctivitis. ACULAR® ophthalmic solution is also indicated for the treatment of postoperative inflammation in patients who have undergone cataract extraction.

Thus, the Levofloxacin as an antibacterial agent and Ketorolac tromethamine as NSAID are individually used in the prior as medications around the time of cataract surgery but there is a need for a combination ophthalmic medicine of Levofloxacin and Ketorolac tromethamine that would be useful for treating or preventing ophthalmic conditions such as bacterial infection and/or inflammation of the eyes.

Thus, there exists a need for topical ophthalmic formulations comprising a combination of fluoroquinolone antibacterial agent such as Levofloxacin and an anti-inflammatory drug such as Ketorolac tromethamine and methods of making and using the same.

But the inventors of present invention have identified an issue of precipitation when the Levofloxacin and Ketorolac tromethamine is mixed together in an ophthalmic formulation. Therefore, the inventors of present invention have stabilized the disclosed formulation by using cyclodextrin as a complexing agent. Further the disclosed compositions of the present invention comprise a complexing agent to stabilize the ophthalmic formulation.

The inventors of the present invention have formulated a topical ophthalmic formulation, wherein when the active ingredients are mixed together with the cyclodextrin as a complexing agent, the active ingredients Levofloxaxcin and Ketorolac tromethamine does not precipitate and stays in solution form.

U.S. Pat. No. 5,324,718 and its continuation, U.S. Pat. No. 5,472,954, both by Thorsteinn Loftsson, provide a method for enhancing the complexation of a cyclodextrin with a lipophilic drug. The use of a water-soluble polymer such as HPMC as a co-solubilizer along with a cyclodextrin is disclosed. It one embodiment the polymer and the cyclodextrin are dissolved in the aqueous medium before the lipophilic drug is added and that solution be maintained from 30° C. to 150° C. for specified periods of time.

U.S. Pat. No. 5,855,916 to Chiesi describes the formation of soluble multicomponent inclusion complexes containing a base type drug, an acid and a cyclodextrin demonstrating enhanced water solubility. Exemplifications in the U.S. Pat. No. 5,855,916 patent include terfenadine, cinnarizine, domperidone, astemizole, ketoconazole, tamoxifene, clomifene and itraconazole as base type drugs.

PCT Publication No. WO02/39993 describes a clear solution or gel preparation of a combination drug comprising an anti-inflammatory agent such as a corticosteroid, an anti-infective agent such as a fluoroquinolone, a complexation-enhancing polymer, and a solubilizer exhibiting an inclusion phenomenon.

### SUMMARY OF THE INVENTION

The present invention provides an ophthalmic pharmaceutical composition comprising a combination of a fluoroquinolone antibacterial agent and an anti-inflammatory agent along with a complexing agent and water, wherein the composition preferably having a between 4 and 8.

An aspect of the present invention is to provide an ophthalmic pharmaceutical composition comprising a fluoroquinolone antibacterial agent such as Levofloxacin and NSAIDs such as Ketorolac tromethamine, optionally with a complexing agent, wherein the composition is useful for topical applications of the eye, ear and/or nose thereof.

Further there is provided a method of treating and/or preventing an ophthalmic condition in a patient, the method comprising topically administering an effective amount of the composition of the present invention to an eye of a patient in need thereof.

In preferred embodiments, the present invention provides a method of prevention and/or reduction of post-operative inflammatory conditions of the eye(s), the method comprising topically administering an effective amount of the composition of the present invention to an eye of a patient in need thereof.

In an aspect of the present invention, the anti-inflammatory agent includes a steroidal or non-steroidal anti-inflammatory agent (NSAID).

Another aspect of the present invention is a method of treating a bacterial infection and/or inflammation of an eye of a patient in need thereof, by topically administering a composition of present invention comprising a combination of levofloxacin and ketorolac tromethamine to the eye, ear or nose of the patients in an amount effective to treat the bacterial infection and/or inflammation.

### DETAILED DESCRIPTION OF THE INVENTION

As used herein, the term “BAC/BKC” wherever appears is an abbreviation for “benzalkonium chloride”.

As used herein, the “CD” wherever appears is an abbreviation for “cyclodextrin”; “SBE7-β-CD” means sulfobutylether7-β-cyclodextrin; “HPCD” means 2-hydroxypropylether-β-cyclodextrin.

As used herein, the “HPMC” means hydroxypropylmethyl cellulose and “PVA” means polyvinyl alcohol.

As used herein, the “NMT” wherever appears is an abbreviation for “not more than”. As used herein, the “RH” wherever appears is an abbreviation for “relative humidity”.

As used herein, the “Pharmaceutically acceptable” wherever appear means that which is generally safe, non-toxic and neither biologically nor otherwise undesirable and includes that which is acceptable for human pharmaceutical use.

Unless indicated otherwise, all ingredient amounts are presented in units of % weight/volume (% w/v).

The term “non-steroidal anti-inflammatory drug” or “NSAID,” as used herein, refers to a class of therapeutic agents with anti-inflammatory actions and the examples of NSAIDs include, but are not limited to, diclofenac, ketorolac tromethamine, bromfenac, nepafenac, their derivatives thereof, and salts thereof.

In one embodiment, the present invention provides an ophthalmic pharmaceutical composition comprising a combination of a fluoroquinolone antibacterial agent and an anti-inflammatory agent along with a complexing agent and water, wherein the composition preferably having a between 4 and 8.

In an aspect of the present invention, the anti-inflammatory agent includes a steroidal or non-steroidal anti-inflammatory agent (NSAID).

In another embodiment, the present invention is to provide an ophthalmic pharmaceutical composition comprising a combination of a fluoroquinolone antibacterial agent such as Levofloxacin and NSAIDs such as Ketorolac tromethamine, optionally with a complexing agent, wherein the composition is useful for topical applications of the eye, ear and/or nose thereof.

In yet another embodiment, the present invention is to provide an ophthalmic pharmaceutical composition comprising essentially of a combination of Levofloxacin and Ketorolac tromethamine, a complexing agent and optionally with pharmaceutical acceptable excipients.

The compositions of the present invention are pharmaceutically acceptable in that they are sterile and suitable for topical or parenteral administration to a patient in need thereof.

In yet another embodiment, there is provided a method of treating and/or preventing an ophthalmic condition in a patient, the method comprising topically administering an effective amount of the composition of the present invention to the eye(s) of a patient in need thereof.

In another embodiment, the present invention provides a method of treating a bacterial infection and/or inflammation of an eye of a patient in need thereof, the method comprising topically administering a combination of levofloxacin and ketorolac tromethamine to the eye, ear or nose of the patients in an amount effective to treat the bacterial infection and/or inflammation.

The present invention provides an ophthalmic compositions for administration to the eye for the treatment or prevention of eye conditions. The presently disclosed ophthalmic compositions of the present eliminate the undesirable side effects that are generally associated with therapeutic ocular compositions.

The presently disclosed ophthalmic compositions of present invention comprises a combination of fluoroquinolone antibacterial agent and a non-steroidal anti-inflammatory drug (NSAID).

In other embodiments, the ophthalmic composition of present invention comprises a combination of a fluoroquinolone antibacterial agent and NSAID optionally along with a complexity agents.

The examples of NSAIDs useful in the present compositions includes, but not limited to, ketorolac tromethamine, bromfenac, diclofenac, nepafenac, their pharmaceutically acceptable equivalents and derivatives thereof and wherein the NSAIDs are present in the concentration range between 0.05% and 5.0% (w/v) (weight/volume) of the compositions.

In particular embodiment, the present compositions comprise ketorolac tromethamine as a sole anti-inflammatory agent.

The fluoroquinolone antibacterial agents that may be used to formulate the present invention includes, but are not limited to, Gatifloxacin, Moxifloxacin, Sitafloxacin, Lomefloxacin, Grepafloxacin, Gemifloxacin, Norfloxacin, Ofloxacin, Levofloxacin, Trovafloxacin, Ciprofloxacin, their derivatives and salts thereof and is present in a concentration range of between 0.05% and 5.0% (w/v) (weight/volume) of the compositions.

In particular embodiment, the present compositions comprise Levofloxacin as a sole fluoroquinolone antibacterial agent.

In preferred embodiments, the present invention provides a method of prevention and/or reduction of post-operative inflammatory conditions of the eye(s), the method comprising topically administering an effective amount of the composition of the present invention to an eye of a patient in need thereof.

The ophthalmic compositions of the present invention are used for the prevention and/or treatment of eye conditions of a patient in need thereof.

The present invention comprises of a combination of a fluoroquinolone antibacterial agent and an anti-inflammatory agent, having the issue of precipitation, stabilized using cyclodextrin as a complexing agent.

In some embodiments, the present invention composition comprises a complexing agent selecting from a group including, but not limited to, naturally occurring cyclodextrins such as α-, β-, and γ-cyclodextrins; modifications of natural occurring cyclodextrins such as, glucosyl-α-cyclodextrin, maltosyl-α-cyclodextrin, glucosyl-β-cyclodextrin, and maltosyl-β-cyclodextrin. Other complexing agents that may be used are 2-Hydroxypropyl-gamma-cyclodextrin, methyl-β-cyclodextrin, hydroxypropyl-β-cyclodextrin, Hydroxypropylbetadex, (2-Hydroxypropyl)-β-cyclodextrin or any derivative thereof and may be used in an amount from about 0.001% (w/v) to about 5.0% (w/v) of the compositions.

Cyclodextrins (CDs), a group of oligosaccharides formed by glucose units bound together in a ring, show a promising ability to form complexes with drug molecules and improve their physicochemical properties without molecular modifications. Cyclodextrins are novel, chemically stable adjuvants that enhance ocular bioavailability of ophthalmic drugs without affecting the barrier function of the eye or increasing the viscosity of the aqueous eye drop formulation. Importantly, since no covalent bonds are formed or broken during the complex formation, the complexes are in dynamic equilibrium with free drug and cyclodextrin molecules. Based on reported literature, it has been suggested that when applied to the eye, HP-β-CD does not pass through the corneal epithelium because the ocular membrane has low affinity for the hydrophilic cyclodextrin; therefore, the cyclodextrin remains in the tear film while the drug partitions into the eye. After the formulation is administered to the eye, the cyclodextrin inevitably ends up in the gastrointestinal tract via the nasolacrimal duct. As per the European Medicines Agency, HP-β-CD even at a concentration of 12.5% (w/v) was well tolerated by the corneal surface of rabbit eyes and was therefore found not to be toxic or irritating in rabbit eyes.

In preferred embodiments, the present invention provides sterile ophthalmic compositions in the form of, but are not limited to, solution, suspension, emulsion, lotions, sprays, ointments, creams, gels, pastes, nanoparticulate systems, drops and the like.

In another preferred embodiment, the present invention provides ophthalmic compositions for topical ophthalmic delivery comprising administering said composition in the eyes. Other preferred embodiments include otic and/or nasal formulations for administration to the ear and/or nose of a human or animal.

In another embodiment, any suitable water soluble polymer may be used to formulate the present invention compositions and includes, but not limited to, polyvinyl alcohol, hydroxypropyl cellulose, hydroxyethyl cellulose, methyl cellulose, hydroxymethyl cellulose, hydroxypropyl methylcellulose, polyvinyl pyrrolidone, polyethylene glycol (Macrogol), polysorbate 80, sodium carboxymethyl cellulose, carboxyvinyl polymer, water-soluble chitosan, sodium chondroitin sulfate, sodium alginate, polyoxyethylene, polyoxypropylene glycol, polyoxyethylene hydrogenated castor oil 40, polyoxyethylene hydrogenated castor oil 60, polyoxy 40 stearate, carboxymethylethylcellulose, carboxyalkyl cellulose esters; starches; pectins such as sodium carboxymethylamylopectin; alginic acid, alkali metal and ammonium salts thereof, carrageenans, galactomannans, traganth, agar-agar, gum arabicum, guar gum and xanthan gum; polyacrylic acids and salts thereof; polymethacrylic acids and salts thereof, including methacrylate copolymers, copolymers of polyvinylpyrrolidone with vinyl acetate; polyalkylene oxides such as polyethylene oxide and polypropylene oxide and copolymers of ethylene oxide and propylene oxide and the like; etc.

In one of the embodiments of the present invention, the polyvinyl alcohol may be used as a viscosity increasing or stabilizing agent. Further it may be used as a lubricating agent for preparing the ophthalmic formulations of the present invention.

In other embodiments, the compositions of the present invention may include additionally tonicity agents and include, but are not limited to, sodium chloride, dextrose, glycerin, mannitol, and potassium chloride and the like.

In another embodiments, the compositions of the present invention may contain a preservative including, but not limited to, benzalkonium chloride (BKC), chlorobutanol, sorbic acid and its salts, alcohol, bronopol, chlorhexidine, imidurea, sodium propionate, benzethonium chloride, phenyl ethanol, phenyl propanol, phenyl mercuric acetate, phenyl mercuric nitrate, phenyl mercuric borate, chlorhexidine acetate or gluconate, cetrimide, chlorocresol, sodium methyl paraben, sodiumpropyl paraben, thimerosaland mixtures thereof, and may be used in an amount from about 0.001% (w/y) to about 0.5% (w/v) by weight of the composition.

In one embodiment, the pH of the compositions are adjusted to between 4 to 8 by adding a pH adjusting agents such as hydrochloric acid, sulfuric acid, nitric acid, carbonic acid, phosphoric acid, citric acid, sodium hydroxide, potassium hydroxide, triethanol amine, tromethanol (tris[hydroxymethyl]aminomethane), meglumine, diethanolamine and the like.

In another embodiments, the suitable chelating agents include, but are not limited to, edetate disodium, edetatetrisodium, edetatetetrasodium, diethyleneaminepentaacetate and mixtures thereof, wherein the chelating agent is generally present in an amount from 0.005-0.2 w/v % relative to the entire composition. The most preferred chelating agent is edetate disodium (EDTA).

In one of another embodiment of the present invention, the sterile, ophthalmic pharmaceutical composition may be aseptically sterilized using membrane filters such as PES (Polyethersulphone), PVDF (Polyvinylidene Fluoride) having pore size of about 0.45 microns to 0.22 microns to filter the composition vehicles.

The terms such as “eye condition” “ophthalmic condition” wherever appears refers to any of a wide variety of pathological ocular conditions or physiologic abnormalities of the eye, such as glaucoma, ocular inflammatory conditions such as keratitis, uveitis, allergy, and dry eye syndrome, ocular infections, ocular allergies, cancerous growth, neo vessel growth originating from the cornea, retinal edema, macular edema, diabetic retinopathy, retinopathy of prematurity, degenerative diseases of the retina (macular degeneration, retinal dystrophies), retinal diseases associated with glial proliferation, and the like.

Non-limiting ophthalmic conditions that are treated with the presently disclosed compositions and methods include, but are not limited to, age related macular degeneration, alkaline erosive keratoconjunctivitis, allergic conjunctivitis, allergic keratitis, anterior uveitis, Behcet's disease, blepharitis, blood-aqueous barrier disruption, chorioiditis, chronic uveitis, conjunctivitis, contact lens-induced keratoconjunctivitis, corneal abrasion, corneal trauma, corneal ulcer, crystalline retinopathy, cystoid macular edema, dacryocystitis, diabetic keratopathy, diabetic macular edema, diabetic retinopathy, dry eye disease, dry age-related macular degeneration, eosinophilic granuloma, episcleritis, exudative macular edema, giant cell arteritis, giant papillary conjunctivitis, glaucoma, glaucoma surgery failure, graft rejection, herpes zoster, inflammation after cataract surgery, iridocorneal endothelial syndrome, iritis, keratoconjunctiva sicca, keratoconjunctival inflammatory disease, keratoconus, necrotic keratitis, neovascular diseases involving the retina, uveal tract or cornea such as neovascular glaucoma, corneal neovascularization, neovascularization following a combined vitrectomy and lensectomy, neovascularization of the optic nerve, and neovascularization due to penetration of the eye or contusive ocular injury, neuroparalytic keratitis, non-infectious uveitisocular herpes, ocular lymphoma, ocular rosacea, ophthalmic infections, ophthalmic pemphigoid, optic neuritis, panuveitis, papillitis, pars planitis, persistent macular edema, phacoanaphylaxis, posterior uveitis, post-operative inflammation, proliferative diabetic retinopathy, proliferative sickle cell retinopathy, proliferative vitreoretinopathy, retinal artery occlusion, retinal detachment, retinal vein occlusion, retinitis pigmentosa, retinopathy of prematurity, rubeosis iritis, scleritis, Stevens-Johnson syndrome, sympathetic ophthalmia, temporal arteritis, thyroid associated ophthalmopathy, uveitis, vernal conjunctivitis, vitamin A insufficiency-induced keratomalacia, vitreitis, and wet age-related macular degeneration.

The bacterial infections of the eye and/or inflammations which may be treated by the compositions of the present invention include, but are not limited to, infections with gram-positive bacteria such as Staphylococcus aureus (including methicillin-susceptible and methicillin-resistant strains), Staphylococcus epidermidis, Streptococcus pneumoniae, Streptococcus (Viridans Group), as well as infections with gram-negative bacteria such as Haemophilus influenzae, Pseudomonas aeruginosa, and Serratiamarcescens.

The terms “treatment,” “treat,” “treating,” and the like, are meant to include slowing or reversing the progression of a disease or disorder. These terms also include alleviating, ameliorating, attenuating, eliminating, or reducing one or more symptoms of a disease or disorder or condition, even if the disease or disorder or condition is not actually eliminated and even if progression of the disease or disorder or condition is not itself slowed or reversed.

The main embodiment of the present invention provides a pharmaceutical composition comprising a combination of a fluoroquinolone antibacterial agent, an anti-inflammatory agent and water, wherein the composition is having a pH between 4 and 8.

In another embodiment of the present invention, wherein the composition further comprising about 0.001% (w/w) to about 5.0% (w/v) by weight of a complexing agent.

In another embodiment of the present invention, wherein the complexing agent is selected from a group including, but not limited to, naturally occurring cyclodextrins such as α-, β-, and γ-cyclodextrins; glucosyl-α-cyclodextrin, maltosyl-α-cyclodextrin, glucosyl-β-cyclodextrin, maltosyl-β-cyclodextrin, 2-Hydroxypropyl-gamma-cyclodextrin, methyl-β-cyclodextrin, hydroxypropyl-β-cyclodextrin, Hydroxypropylbetadex, (2-Hydroxypropyl)-β-cyclodextrin or any derivative thereof.

In another embodiment of the present invention, wherein the fluoroquinolone antibacterial agent is present in a concentration range of (1.05% to 5.0% by weight of the composition.

In another embodiment of the present invention, wherein the fluoroquinolone antibacterial agent is selected from the group consisting of, but are not limited to, Gatifloxacin, Moxifloxacin, Sitafloxacin, Lomefloxacin, Grepafloxacin, Gemifloxacin, Norfloxacin, Ofloxacin, Levofloxacin, Trovafloxacin, Ciprofloxacin, their derivatives and salts thereof.

In another embodiment of the present invention, wherein the anti-inflammatory agent includes a non-steroidal anti-inflammatory agent (NSAID).

In another embodiment of the present invention, wherein the non-steroidal anti-inflammatory agent is selected from the group includes, but not limited to, ketorolac tromethamine, bromfenac, diclofenac, nepafenac, their pharmaceutically acceptable equivalents and derivatives thereof.

In another embodiment of the present invention, wherein the anti-inflammatory agent is present in a concentration range of 0.05% to 5.0% by weight of the composition.

In another embodiment of the present invention, wherein the composition further comprising about 0.001% (w/v) to about 0.5% (w/v) by weight of a preservative.

In another embodiment of the present invention, wherein the preservative is selected from the group consisting of, but not limited to, benzalkonium chloride (BKC), chlorobutanol, sorbic acid and its salts, alcohol, bronopol, chlorhexidine, imidurea, sodium propionate, benzethonium chloride, phenyl ethanol, phenyl propanol, phenyl mercuric acetate, phenyl mercuric nitrate, phenyl mercuric borate, chlorhexidine acetate or gluconate, cetrimide, chlorocresol, thimerosal and mixtures thereof.

In another embodiment of the present invention, wherein the composition further comprising a water soluble polymer.

In another embodiment of the present invention, wherein the water soluble polymer is selected from the group consisting of polyvinyl alcohol, hydroxypropylmethylcellulose, methylcellulose, carboxymethyl cellulose, polyvinylpyrrolidone, and poloxamer or mixtures thereof.

In another embodiment of the present invention, wherein the composition additionally comprises pharmaceutical acceptable excipients.

In another embodiment of the present invention, wherein the pharmaceutically acceptable excipients are selected from group comprising a tonicity agent, a viscosity increasing or stabilizing agent, a pH adjusting agent, a chelating agent, a preservative, or a combination of two or more thereof.

In another embodiment of the present invention, wherein the composition is sterile and suitable for topical, ophthalmic or parenteral administration to the eye(s) of a patient in need thereof.

In another embodiment of the present invention, wherein the composition is administered either once a day, twice a day or thrice a day to each eye in need thereof.

In another embodiment of the present invention, wherein both the fluoroquinolone antibacterial agent, and an anti-inflammatory agent is present in dissolved form.

In another embodiment of the present invention, wherein the composition is in the form of an aqueous solution suitable for ophthalmic use.

Yet another embodiment of the present invention provides an aqueous pharmaceutical composition comprising:
0.05% to 5.0% by weight of the composition of a fluoroquinolone antibacterial active agent;
0.05% to 5.0% by weight of the composition of an anti-inflammatory agent;
0.001% (w/v) to about 5.0% (w/v) by weight of complexing agent;
water soluble polymer,
water,
wherein the composition is an ophthalmic solution, having a pH between 4 and 8.

In another embodiment of the present invention, wherein the composition further comprising about 0.001% (w/v) to about 0.5% (v/v) by weight of a preservative.

In another embodiment of the present invention, wherein the composition additionally comprises pharmaceutically acceptable excipients.

Yet another embodiment of the present invention provides a method of treating a bacterial infection of an eye and/or inflammation of a subject in need thereof, comprising topically administering an effective amount of the composition to the eye of said subject to treat said bacterial infection.

Yet another embodiment of the present invention provides a method of treating and/or preventing an ophthalmic condition of a subject in need thereof, the method comprising topically administering an effective amount of the composition to the eye(s) of a patient in need thereof.

Yet another embodiment of the present invention provides a method of prevention and/or reduction of post-operative inflammatory conditions of the eye(s), the method comprising topically administering an effective amount of the composition to an eye of a patient in need thereof.

Yet another embodiment of the present invention provides a method of treating a bacterial infection and/or inflammation of an eye of a patient in need thereof, the method comprising topically administering a combination of levofloxacin and ketorolac tromethamine to the eye(s) of the patients in an amount effective to treat the bacterial infection and/or inflammation.

Yet another embodiment of the present invention provides a method of treating and/or preventing an ophthalmic condition of an eye of a patient in need thereof, the method comprising topically administering a combination of levofloxacin and ketorolac tromethamine to the eye(s) of the patients in an amount effective to treat and/or prevent an ophthalmic condition.

Yet another embodiment of the present invention provides use of a composition for the treatment of a bacterial infection of an eye and/or inflammation in a subject.

### EXAMPLES

The scope of the present invention s illustrated by the following examples as disclosed herein which are not meant to restrict the scope of the invention in any manner whatsoever.

The term ‘q.s.’ wherever appears in the examples is an abbreviation for ‘quantity sufficient’ which is the amount of the excipient in such quantities that is just sufficient for its use in the composition of the present invention.

### Example 1

| Ingredients | Concentration Range % (w/v) |
|---|---|
| Levofloxacin hemihydrate* | 0.05-5.00 |
| Ketorolac tromethamine | 0.05-5.00 |
| Benzalkonium chloride | 0.001-0.50 |
| Hydroxypropylbetadex | 0.001-5.00 |
| Disodium edetate | 0.005-0.5 |
| Sodium chloride | 0.05-2.0 |
| Hydrochloric acid | Adjust to pH 4 to 8 |
| Sodium Hydroxide | Adjust to pH 4 to 8 |
| Water | q.s. to 100% |

*Levofloxacin hemihydrate equivalent to Levofloxacin 5 mg/mL

### Example 2

| Ingredients | Concentration Range % (w/v) |
|---|---|
| Levofloxacin hemihydrate* | 0.05-5.0 |
| Ketorolac tromethamine | 0.05-5.0 |
| Benzalkonium chloride | 0.001-0.5 |
| Hydroxypropylbetadex | 0.001-5.0 |
| Polyvinyl alcohol 26-88 | 0.05-3.00 |
| Disodium edetate | 0.005-0.5 |
| Sodium chloride | 0.05-2.0 |
| Hydrochloric acid | Adjust to pH 4 to 8 |
| Sodium Hydroxide | Adjust to pH 4 to 8 |
| Water | q.s. to 100% |

*Levofloxacin hemihydrate equivalent to Levofloxacin 5 mg/mL

### Example 3

| | Concentration | Concentration |
|---|---|---|
| Ingredients | (% w/v) | (mg/mL) |
| Levofloxacin hemihydrate* | 0.5 | 5.000 |
| Ketorolac tromethamine | 0.5 | 5.000 |
| Benzalkonium chloride | 0.01 | 0.100 |
| Hydroxypropylbetadex | 1.949 | 19.490 |
| Disodium edetate | 0.1 | 1.000 |
| Sodium chloride | 0.77 | 7.700 |
| Hydrochloric acid | Adjust to pH 7.3 | Adjust to pH 7.3 |
| Sodium Hydroxide | Adjust to pH 7.3 | Adjust to pH 7.3 |
| Water | q.s. to 100% | q.s. to 1 mL |

*Levofloxacin hemihydrate equivalent to Levofloxacin 5 mg/mL

### Stability Study

Stability studies were conducted in three-piece white opaque LDPE bottle at accelerated condition (40° C.±2° C./Not more than 25% RH) for 6 months and the stability data of Example 3 is summarized in below Table 1.

TABLE 1
Stability Study of Levofloxacin 0.5% and Ketorolac Tromethamine 0.5% eye drops.
Levofloxacin 0.5% and Ketorolac Tromethamine 0.5% eye drops
(5 mL in 5 mL three-piece white opaque LDPE bottle)

| | Condition: 40° C. ± 2° C./NMT 25% RH | | | |
|---|---|---|---|---|
| Test Parameters | Initial | 1 Month | 3 Month | 6 Month |
| Appearance | Slight greenish | Slight greenish | Slightly greenish | Greenish |
| | yellow colored, | yellow colored, | yellow colored | yellow colored |
| | clear solution | clear solution | clear solution | clear solution |
| pH | 7.10 | 7.16 | 7.26 | 7.08 |
| Osmolality (mOsmol/kg) | 304 | 304 | 308 | 311 |
| Assay of Levofloxacin (%) | 96.8 | 98.4 | 99.6 | 101.3 |
| Assay of Ketorolac | 100.0 | 99.1 | 101.6 | 103.5 |
| Tromethamine (%) | | | | |
| Content of Benzalkonium | 100.7 | 101.0 | 103.6 | 104.0 |
| Chloride (%) | | | | |

Related substances
Related substances of levofloxacin (%)

| | | | | |
|---|---|---|---|---|
| Impurity- C (N-Oxide) | 0.05 | 0.02 | 0.04 | 0.03 |
| Any unspecified | BDL | BDL | BDL | BDL |
| individual Impurity | | | | |

Related Substances of Ketorolac Tromethamine (%)

| | | | | |
|---|---|---|---|---|
| Impurity- A | ND | BQL | ND | ND |
| Impurity- B | 0.03 | 0.01 | 0.01 | 0.02 |
| Impurity- C | ND | ND | ND | ND |
| Impurity- D | ND | ND | ND | ND |
| Any unspecified | BDL | BDL | BDL | BDL |
| individual Impurity | | | | |
| Total impurities | 0.08 | 0.03 | 0.05 | 0.05 |

ND: Not detected
NMT: Not more than
BQL: Below qualification limit
BDL: Below disregard limit

### Conclusion

The results of the stability study indicate that no significant change in Description, assay of levofloxacin, assay of Ketorolac Tromethamine, content of BKC, pH, Osmolality, and related substances were observed, when stored at a condition of 40° C.±2° C./NMT 25% RH for 6 months. Hence, it is concluded that the Levofloxacin 0.5% and Ketorolac Tromethamine 0.5% eye drops are stable under accelerated and long term condition.

### Freeze Thaw/Thermal Cycling and Short Term Excursion Study

Levofloxacin 0.5% and Ketorolac Tromethamine 0.5% Eye drops samples were exposed to Freeze thaw/thermal cycling and short term excursion studies at different temperature fluctuations with a number of cycles; to find out the effect of temperature stress on the stability of the drug product.

### Freeze Thaw/Thermal Cycling Study

Samples from Example 3 of Levofloxacin 0.5% and Ketorolac Tromethamine 0.5% Eye drops, filled in 5 mL white opaque low density polyethylene (LDPE) bottle with opaque LDPE nozzle and white colored high density polyethylene (HDPE) cap, overwrapped in R&D label and supplied in R&D unit carton along with package insert for freeze thaw/thermal study were frozen (at about −20° C.) for 2 days and the same samples were then thawed at 40° C.±2° C./NMT 25% RH for 2 days as a one cycle.
1^{st} Cycle: First 2 days in freezer (at about −20° C.) and thawed only once at 40° C.±2° C./NMT 25% RH for 2 days.
2^{nd} Cycle: Continuation of 1^{st} Cycle at freezer (at about −20° C. for 2 days) and thawed twice at 40° C.±2° C./NMT 25% RH for 2 days.
3^{rd} Cycle: Cycle: Continuation of 2^{nd} Cycle at freezer (at about −20° C. for 2 days) and thawed thrice at 40° C.±2° C./NMT 25% RH for 2 days.
The same process was continued for 3 cycles and at each cycle samples were analysed for appearance, pH, color, clarity, osmolality, assay of Levofloxacin and Ketorolac Tromethamine and related substances and the results are summarized in below Table 2.

TABLE 2
Results of Freeze thaw/Thermal cycling
of Example 3.5 mL in 5 mL LDPE vial

| | Conditions | | | |
|---|---|---|---|---|
| | Freeze thaw/Thermal cycling | | | |
| Test parameter | Initial | 1^{st} cycle | 2^{nd} cycle | 3^{rd} cycle |
| Appearance | # | # | # | # |
| pH | 7.10 | 7.13 | 7.11 | 7.11 |
| Osmolality (mOsmol/kg) | 304 | 302 | 303 | 303 |
| Assay of Levofloxacin (%) | 96.8 | 96.6 | 96.8 | 96.6 |
| Assay of Ketorolac (%) | 100.0 | 100.4 | 100.6 | 100.2 |
| BKC Content (%) | 100.7 | 100.2 | 100.1 | 99.9 |

Related substances of Levofloxacin (%)

| | | | | |
|---|---|---|---|---|
| Impurity- C (N-Oxide) | 0.05 | 0.02 | 0.02 | 0.02 |
| Any unspecified | BDL | BDL | BDL | BDL |
| individual Impurity | | | | |

Related substances of Ketorolac (%)

| | | | | |
|---|---|---|---|---|
| Ketorolac Impurity- A | ND | ND | ND | ND |
| Ketorolac Impurity- B | 0.03 | 0.01 | 0.01 | 0.01 |
| Ketorolac Impurity- C | ND | ND | ND | ND |
| Ketorolac Impurity- D | ND | ND | ND | ND |
| Any unspecified | BDL | BDL | BDL | BDL |
| individual Impurity | | | | |
| Total Impurities | 0.08 | 0.03 | 0.03 | 0.03 |

ND: Not detected
NMT: Not more than
BDL: Below disregard limit
# Slightly greenish yellow colored clear solution

### Conclusion

The above results showed that all the quality parameters of the treated product samples are compared with untreated (Initial) sample and the test results were well within the acceptable ranges and no significant change was observed at different condition for all testing parameters.

### Short Term Excursion Study

Samples from Example 3 of Levofloxacin 0.5% and Ketorolac Tromethamine 0.5% Eye drops, filled in 5 mL white opaque low density polyethylene (LDPE) bottle with opaque LDPE nozzle and white colored high density polyethylene (HDPE) cap, overwrapped in R&D label and supplied in R&D unit carton along with package insert for short term excursion study were frozen (at about −20° C.) for 2 days and the same samples were then exposed at stress condition of 50° C.±2° C. 2 days as a one cycle and the results are summarised in Table 3.

TABLE 3
Results of short term Excursion study, 5 mL in 5 mL LDPE vial

Conditions
Short term Excursion study

| | | After exposure |
|---|---|---|
| Test parameter | Initial | of 1 cycle |
| Appearance | Slight greenish | Slight greenish |
| | yellow colored, | yellow colored, |
| | clear solution | clear solution |
| pH | 7.10 | 7.14 |
| Osmolality (mOsmol/kg) | 304 | 313 |
| Assay of Levofloxacin (%) | 96.8 | 97.3 |
| Assay of Ketorolac (%) | 100.0 | 100.9 |
| BKC Content (%) | 100.7 | 100.0 |

Related substances of Levofloxacin (%)

| | | |
|---|---|---|
| Impurity- C (N-Oxide) | 0.05 | 0.02 |
| Any unspecified | BDL | BDL |
| individual Impurity | | |

Related substances of Ketorolac (%)

| | | |
|---|---|---|
| Ketorolac Impurity- A | ND | ND |
| Ketorolac Impurity- B | 0.03 | 0.01 |
| Ketorolac Impurity- C | ND | ND |
| Ketorolac Impurity- D | ND | BDL |
| Any unspecified | BDL | BDL |
| individual impurity | | |
| Total Impurities | 0.08 | 0.03 |

ND: Not detected
NMT: Not more than
BDL: Below disregard limit

### Conclusion

The above results showed that all the quality parameters of the treated product samples are compared with untreated (Initial) sample and the test results were well within the acceptable ranges and no significant change was observed at different condition for all testing parameters.

### Photo Stability Studies

The samples of Levofloxacin 0.5% and Ketorolac Tromethamine 0.5% Eye drops were subjected to photo stability study in different packs such as:
1. Immediate pack (5 mL in 5 mL LDPE vial overwrapped in R&D label—without aluminium pouch and carton)
2. Market pack (5 mL in 5 mL LDPE vial overwrapped in R&D label and supplied in R&D unit carton along with package insert).
3. Control (Aluminum wrapped immediate Pack)
   The results were compared with initial analysis and also with control sample. Samples of Levofloxacin 0.5% and Ketorolac Tromethamine 0.5% Eye drops were exposed simultaneously to 200 watt-hours/m² of near UV light and 1.2 million Lux hours of cool fluorescent light in a photo stability chamber. The samples exposed for photo stability were evaluated and the results are summarized in Table 4.

TABLE 4
Results of Photo stability studies.

| | Photo stability studies | | | |
|---|---|---|---|---|
| Test | | Control Aluminium | Immediate | Market |
| parameter | Initial | Pack | Pack | Pack |
| Appearance | Slight greenish | Clear, Slightly | Clear, Slightly | Clear, Slightly |
| | yellow colored, | Greenish yellow | Greenish yellow | Greenish yellow |
| | clear solution | color solution | color solution | color solution |
| pH | 7.10 | 7.21 | 7.19 | 7.23 |
| Osmolality (mOsmol/kg) | 304 | 300 | 301 | 301 |
| Color of solution | Sample solution was | Sample solution is | Sample solution is | Sample solution is |
| | more intensely | more intensely | more intensely | more intensely |
| | colored than | colored than the | colored than | colored than |
| | GY5 and | GY₆ but | the GY₆ but | the GY₆ but |
| | less intensely | less intensely | less intensely | less intensely |
| | colored than | colored than | colored than | colored than |
| | GY4 | GY₅ | GY₅ | GY₅ |
| Clarity By UV (at 650 nm) | Not performed | % Transmittance | % Transmittance | % Transmittance |
| | | value obtained from | value obtained from | value obtained from |
| | | the sample solution | the sample solution | the sample solution |
| | | is more than % | is more than % | is more than % |
| | | transmittance value | transmittance value | transmittance value |
| | | obtained from the | obtained from the | obtained from the |
| | | reference suspension | reference suspension | reference suspension |
| | | I at 650 nm | I at 650 nm | I at 650 nm |
| Assay of Levofloxacin (%) | 96.8 | 98.0 | 96.8 | 98.1 |
| Assay of Ketorolac (%) | 100.0 | 99.5 | 97.8 | 99.5 |
| BKC Content (%) | 100.7 | 98.9 | 99.2 | 99.4 |

Related substances of Levofloxacin (%)

| | | | | |
|---|---|---|---|---|
| Levofloxacin-N-Oxide | 0.05 | 0.02 | 0.21 | 0.02 |
| Any unspecified | BDL | BDL | 0.17 (RRT: | BDL |
| individual impurity | | | 0.39) | |

Related substances of Ketorolac (%)

| | | | | |
|---|---|---|---|---|
| Impurity A | ND | ND | 0.04 | ND |
| Impurity B | 0.03 | 0.01 | 0.18 | 0.01 |
| Impurity C | ND | ND | 0.01 | ND |
| Impurity D | ND | 0.01 | 0.01 | 0.01 |
| Any unspecified | BDL | BDL | 0.12(RRT: | BDL |
| individual impurity | | | 0.20) | |
| Total Impurities | 0.08 | 0.92 | 1.85 | 0.84 |

ND: Not detected
NMT: Not more than
BDL: Below disregard limit
RRT: Relative Retention Time

### Conclusion

The results of the immediate pack, market pack and control sample were within the set product specifications which clearly indicates that the product is photostable in the proposed pack configuration. However, as there is slight increase of related substances in immediate pack; the product is recommended to store in the marketed pack during storage with an instruction of protect from light as a precautionary measure.

### Summary of Non-Clinical Study of a Combination Product of Levofloxacin+Ketorolac Eye Drops

A comparative nonclinical study of subchronic toxicity and local irritating effect of the investigational medicinal product, Levofloxacin+ketorolac, 0.5%+0.5%, eye drops (Sentiss Pharma Pvt. Ltd., India) and the reference monodrugs, Oftaquicks®, eye drops, 0.5% (Santen JSC, Finland) and Elipa®, eye drops, solution, 0.5% (Laboratorio Edol Produtos Pharmaceuticos S.A., Portugal) was performed.
The subchronic toxicity was studied using the male rabbits of the Soviet Shinshilla breed. The investigational medicinal products were injected in the conjunctiva on a daily basis, for 14 days, in the following doses: 0.04 ml/kg (single equitherapeutic dose) and 0.4 ml/kg (10-times equitherapeutic dose) for the investigational medicinal product and in the major dose of 0.4 ml/kg for the reference mono drugs.

### Goal and Objectives Study Scope

This study is aimed at the nonclinical safety study of the investigational medicinal product, Levofloxacin+ketorolac, 0.5%+0.5%, eye drops (Sentiss Pharma Pvt. Ltd., India).
This study included the equivalence check of subchronic toxicity and local irritating effect of the investigational medicinal product, Levofloxacin+ketorolac, 0.5%+0.5%, eye drops (Sentiss Pharma Pvt. Ltd., India) and the reference monodrugs, Oftaquicks®, eye drops, 0.5% (Santen JSC, Finland) and Elipa®, eye drops, solution, 0.5% (Laboratorio Edol Produtos Pharmaceuticos S.A., Portugal).
The investigational medicinal product is a combined medicine represented by a fixed combination of the active ingredients authorized in at least one of the Eurasian Economic Union member states as a single-component medicinal product but not administered in this combination as authorized medicinal products. The comparative study of the 14-day sub-chronic toxicity and local irritating effect of the investigational medicinal product vs the mono drugs included into that combination.
In the sub-chronic toxicity study, the investigational drugs were administered into the conjunctiva QD in both eyes of rabbits, for 14 days.
The local irritating effect of the drugs in conjunctival administration was studied, together with the sub-chronic toxicity study.

### Sub-Chronic Toxicity

The investigational medicinal product was studied in parallel with reference medicinal products: Oftaquicks®, eye drops, 0.5% (Santen JSC, Finland) and Elipa®, eye drops, solution, 0.5% (Laboratorio Edol Produtos Pharmaceuticos S.A., Portugal). The subchronic toxicity was studied in one species of animals (male rabbits), in two doses for the investigational medicinal product and a major dose for the reference medicinal monodrugs only. The test dose of the investigational or reference medicinal product was administered to each animal Q.D. in the conjunctiva for 14 days. In the control animal group, the investigational drugs were substituted for a sterile 0.9% sodium chloride solution.
During the study, the integral animal health indicators were recorded: appearance (daily), body weight changes, food activity, and rectal body temperature (weekly). The functional status indicators of the excretory system were estimated once, at the end of the experiment. 14 days after the experiment started, the peripheral blood samples were taken for hematological and biochemical studies, and then the animals were subjected to euthanasia. After euthanasia, the pathological autopsy was performed, the organ weights were determined, materials for autopsy (fixation of organs and tissues in 10% formalin) and histological cuts were prepared.

### Local Irritating Effect

As part of the study of the general toxic effect of the investigational medicinal products, the local irritating effect in the case of the conjunctival administration was studied.
The ophthalmic toxicity indicators, which were estimated using the external examination and the eyeball histological examination, served as the criterion of local irritating effect.

### Visual Assessment

As part of the daily clinical examination of the animals, the intensity of the local irritating effect was assessed when the investigational medicinal products were injected into the eyes, using the rating scale. The eye condition assessment time ranges from 10 to 20 minutes after the administration of the investigational medicinal products.

### Study Findings

The following conclusions were made as a result of the sub-chronic toxicity study:
1) In the case of 14-day administration of the investigational medicinal product at 0.04 ml/kg and 0.4 ml/kg, and of the reference medicinal monoproducts, at 0.4 ml/kg, no death was recorded.
2) No reliable intergroup differences were found in the feed and water consumption indicators.
3) No reliable intergroup differences in the body weight changes were established.
4) No significant intergroup differences for the rectal body temperature indicators were found.
5) No significant intergroup differences were found for the relative weight of the organs.
6) No reliable intergroup differences were found in the biochemical blood serum indicators.
7) No significant intergroup differences were found in the hematological blood parameters and the leukocyte formula.
8) When the functional condition of the kidneys was studied, no significant intergroup differences in such indicators as the protein urine content, relative density and urine pH.
9) As the autopsy findings suggested, no morphological changes in the liver, spleen, stomach, small and large intestines, the pancreas, thymus, kidneys, adrenals, heart, lungs, testis and cerebral tissues under the impact of the investigational medicinal product and the reference medicinal monoproducts were found in the subchronic toxicity experiments.
10) No cytomorphological changes in the liver, kidneys, adrenals, thymus, spleen, pancreas, stomach, small and large intestines, heart, lungs, testis and the cerebral tissue under the impact of the investigational medicinal product develop.
   The following conclusions were made as a result of the local irritating effect study:
1. Neither the investigational medicinal product nor the reference medicinal monoproducts have any significant local irritating effect after the multiple administration, both in the single-dose for the investigational medicinal product and in the 10-times dose for the investigational medicinal product and the reference medicinal monoproducts, as testified by the assessment of ophthalmotoxity indicators.

### Conclusion

Based on the findings of the comparative nonclinical study of sub-chronic toxicity and local irritating effect of the investigational medicinal product, Levofloxacin+ketorolac, 0.5%+0.5%, eye drops (Sentiss Pharma Pvt. Ltd., India) and the reference mono drugs, Oftaquicks®, eye drops, 0.5% (Santen JSC, Finland) and Elipa®, eye drops, solution, 0.5% (Laboratorio Edol Produtos Pharmaceuticos S.A., Portugal), which were obtained using the biochemical, hematological and histological study methods, it is concluded that the toxicity of the investigational medicinal product is equivalent to that of reference mono drugs.

### UTILITY OF THE PRESENT INVENTION

The present inventors have provided an ophthalmic composition. The composition comprises a combination of fluoroquinolone antibacterial agent and an anti-inflammatory agent along with a complexing agent and water. The composition has shown potential effects in treating and/or preventing an ophthalmic condition in a patient.

1. A pharmaceutical composition comprising a combination of a fluoroquinolone antibacterial agent, an anti-inflammatory agent and water, wherein the composition is having a pH between 4 and 8.

2. The composition as claimed in claim 1, wherein the composition further comprising about 0.001% (w/v) to about 5.0% (w/v) by weight of a complexing agent.

3. The composition as claimed in claim 2, wherein the complexing agent is selected from a group including, but not limited to, naturally occurring cyclodextrins such as α-, β-, and γ-cyclodextrins; glucosyl-α-cyclodextrin, maltosyl-α-cyclodextrin, glucosyl-β-cyclodextrin, maltosyl-β-cyclodextrin, 2-Hydroxypropyl-gamma-cyclodextrin, methyl-β-cyclodextrin, hydroxypropyl-β-cyclodextrin, Hydroxypropylbetadex, (2-Hydroxypropyl)-β-cyclodextrin or any derivative thereof.

4. The composition as claimed in claim 1, wherein the fluoroquinolone antibacterial agent is present in a concentration range of 0.05% to 5.0% by weight of the composition.

5. The composition as claimed in claim 1, wherein the fluoroquinolone antibacterial agent is selected from the group consisting of, but are not limited to, Gatifloxacin, Moxifloxacin, Sitafloxacin, Lomefloxacin, Grepafloxacin, Gemifloxacin, Norfloxacin, Ofloxacin, Levofloxacin, Trovafloxacin, Ciprofloxacin, their derivatives and salts thereof.

6. The composition as claimed in claim 1, wherein the anti-inflammatory agent includes a non-steroidal anti-inflammatory agent (NSAID).

7. The composition as claimed in claim 6, wherein the non-steroidal anti-inflammatory agent is selected from the group includes, but not limited to, ketorolac tromethamine, bromfenac, diclofenac, nepafenac, their pharmaceutically acceptable equivalents and derivatives thereof.

8. The composition as claimed in claim 1, wherein the anti-inflammatory agent is present in a concentration range of 0.05% to 5.0% by weight of the composition.

9. The composition as claimed in claim 1, wherein the composition further comprising about 0.001% (w/v) to about 0.5% (w/v) by weight of a preservative.

10. The composition as claimed in claim 9, wherein the preservative is selected from the group consisting of, but not limited to, benzalkonium chloride (BKC), chlorobutanol, sorbic acid and its salts, alcohol, bronopol, chlorhexidine, imidurea, sodium propionate, benzethonium chloride, phenyl ethanol, phenyl propanol, phenyl mercuric acetate, phenyl mercuric nitrate, phenyl mercuric borate, chlorhexidine acetate or gluconate, cetrimide, chlorocresol, thimerosal and mixtures thereof.

11. The composition as claimed in claim 1, wherein the composition further comprising a water soluble polymer.

12. The composition as claimed in claim 11, wherein the water soluble polymer is selected from the group consisting of polyvinyl alcohol, hydroxypropylmethylcellulose, methylcellulose, carboxymethyl cellulose, polyvinylpyrrolidone, and poloxamer or mixtures thereof.

13. The composition as claimed in claim 1, wherein the composition additionally comprises pharmaceutical acceptable excipients.

14. The composition as claimed in claim 13, wherein the pharmaceutically acceptable excipients are selected from group comprising a tonicity agent, a viscosity increasing or stabilizing agent, a pH adjusting agent, a chelating agent, a preservative, or a combination of two or more thereof.

15. The composition as claimed in claim 1, wherein the composition is sterile and suitable for topical, ophthalmic or parenteral administration to the eye(s) of a patient in need thereof.

16. The composition as claimed in claim 1, wherein the composition is administered either once a day, twice a day or thrice a day to each eye in need thereof.

17. The composition as claimed in claim 1, wherein both the fluoroquinolone antibacterial agent, and an anti-inflammatory agent is present in dissolved form.

18. The composition as claimed in claim 1, wherein the composition is in the form of an aqueous solution suitable for ophthalmic use.

19. An aqueous pharmaceutical composition comprising:
0.05% to 5.0% by weight of the composition of a fluoroquinolone antibacterial active agent;
0.05% to 5.0% by weight of the composition of an anti-inflammatory agent;
0.001% (w/v) to about 5.0% (w/v) by weight of complexing agent;
water soluble polymer,
water,
wherein the composition is an ophthalmic solution, having a pH between 4 and 8.

20. The composition as claimed in claim 1, wherein the composition further comprising about 0.001% (w/v) to about 0.5% (w/v) by weight of a preservative.

21. The composition as claimed in claim 1, wherein the composition additionally comprises pharmaceutically acceptable excipients.

22. A method of treating a bacterial infection of an eye and/or inflammation of a subject in need thereof, comprising topically administering an effective amount of the composition as claimed in claim 1 to the eye of said subject to treat said bacterial infection.

23. A method of treating and/or preventing an ophthalmic condition of a subject in need thereof, the method comprising topically administering an effective amount of the composition as claimed in claim 1 to the eye(s) of a patient in need thereof.

24. A method of prevention and/or reduction of post-operative inflammatory conditions of the eye(s), the method comprising topically administering an effective amount of the composition as claimed in claim 1 to an eye of a patient in need thereof.

25. A method of treating a bacterial infection and/or inflammation of an eye of a patient in need thereof, the method comprising topically administering a combination of levofloxacin and ketorolac tromethamine to the eye(s) of the patients in an amount effective to treat the bacterial infection and/or inflammation.

26. A method of treating and/or preventing an ophthalmic condition of an eye of a patient in need thereof, the method comprising topically administering a combination of levofloxacin and ketorolac tromethamine to the eye(s) of the patients in an amount effective to treat and/or prevent an ophthalmic condition.

27. Use of a composition as claimed in claim 1 for the treatment of a bacterial infection of an eye and/or inflammation in a subject.
